(19) United States
(12) Patent Application Publication
(10) Pub. No.: US 2003/0186890 A1
(43) Pub. Date: Oct. 2, 2003

(54) Amphipathic linear peptides and formulations containing said peptides

(75) Inventors: Guillaume Drin, Montpellier (FR); Jerome Gomar, Uchaud (FR); Jamal Temsamani, Nimes (FR); Anthony R. Rees, Saint-Chaptes (FR)

Correspondence Address: IP DEPARTMENT OF PIPER RUDNICK LLP, 3400 TWO LOGAN SQUARE, 18TH AND ARCH STREETS, PHILADELPHIA, PA, 19103, US

(73) Assignee: SYNT:EM S.A., NIMES (FR)

(21) Appl. No.: 10/336,312

(22) Filed: Jan. 3, 2003

Related U.S. Application Data
(63) Continuation of application No. PCT/FR01/02129, filed on Jul. 3, 2001.

(30) Foreign Application Priority Data
Jul. 3, 2000 (FR) 00/08633

(51) Int. Cl. A61K 38/10; A61K 38/08; C07K 7/08; C07K 7/06

(52) U.S. Cl. 514/14; 514/15; 530/326; 530/327

(57) ABSTRACT

Peptides containing or composed of an antibiotic peptide derivative by (i) modifying the cysteine residues such that the peptide is free of disulphide bridges, (ii) substituting 1 to 18 and, preferentially, 1 to 6 amino acids and/or permuting at least one amino acid pair, the substitutions and/or permutation being such that the peptide is amphipathic in nature, and a compound formed from at least one of the peptides bound directly or indirectly to at least one active substance.

### RELATED APPLICATION

This is a continuation of PCT International Appln. No. PCT/FR01/02129 filed Jul. 3, 2001 which claims benefits from French Application No. 00/08633 filed Jul. 3, 2000.

1. Field of the Invention

This invention relates to linear peptides and their use to carry active substances. More particularly, the invention relates to highly amphipathic linear peptides derived from antibiotic peptides or their analogues. The invention also relates to new compounds formed from an amphipathic linear peptide bound with at least one active substance and the preparation of said compounds and formulations containing said compounds.

2. Background

The problem of introducing different substances with pharmacological properties into live cells is of major interest for research and therapeutic and diagnostic purposes.

One of the priorities of work in this field is to find effective means to increase the penetration efficacy of active substances, such as conventional molecules, peptides, proteins, or nucleic acids such as oligonucleotides, into live cells.

Several strategies have been proposed to enable or increase the passage of said substances through the cell membrane. Among said strategies, we have developed a system to carry active substances through the cell membrane using carrier peptides. This carrying strategy offer numerous advantages, since the carrier peptide can be synthesised chemically and most of the above-mentioned active substances can be bound with the carrier in a simple and effective manner.

Protegrin and tachyplesin are natural peptides with a hairpin type structure held by disulphide bridges. Said bridges play an important role in the cytolytic activity observed on human cells. Research work on said peptides led to the discovery that an irreversible reduction of said disulphide bridges makes it possible to generate linear peptides which have the property of passing rapidly through cell membranes. The linear peptides and their use as carriers for active substances are described in PCT international patent application No. W099/07728.

We then attempted to define new amino acid sequences capable of acting as an internalisation and addressing carrier for active substances and several peptides with enhanced physico-chemical properties were synthesised for this purpose.

### SUMMARY OF THE INVENTION

This invention relates to a peptide comprising an antibiotic peptide derivative formed by:

modifying the cysteine residues such that the peptide is free of disulphide bridges, and

substituting 1 to 18 amino acids and/or permuting at least one amino acid pair, the substitutions and/or permutation being such that the peptide is amphipathic in nature, and has a mean hydrophobicity per residue <H> between 0.15 and 0.7.

The invention also relates to a compound formed from the above peptide bound directly or indirectly to at least one active substance.

The invention further relates to a pharmaceutical formulation comprising as an active agent a therapeutically effective quantity of at least one compound described above in an acceptable vehicle.

### BRIEF DESCRIPTION OF THE DRAWING

FIG. 1 is a graph of the vectorisation of doxorubicin with cector SynB1/3Cit.

### DETAILED DESCRIPTION

This invention relates to a linear peptide containing or composed of an antibiotic peptide derivative by:

modifying the cysteine residues such that said peptide is free of disulphide bridges,

substituting 1 to 18 and, preferentially, 1 to 6 amino acids and/or permutation of at least one amino acid pair, said substitutions and/or permutation being such that said peptide is amphipathic in nature.

The term antibiotic peptides refers to peptides with more than 5 to 7 amino acids showing cytolytic activity on human or animal cells. These peptides are natural peptides or synthetic peptides and fragments of said peptides. The invention particularly envisages peptides derived from protegrin and tachyplesin, an analogue or a fragment of said peptides.

The peptides according to the invention are free of disulphide bridges such as those described for example in published PCT international patent application No. W099/07728. The absence of disulphide bridges in the peptides according to the invention may be obtained using any technique known to those of ordinary skill in the art, particularly by eliminating or replacing cysteine residues by other amino acids or by inhibiting −SH groups such that they no longer form disulphide bridges.

The amphipathic nature of the peptides according to the invention was expressed by the value of the mean hydrophobicity per residue <H> and the helical hydrophobic moment <μH>α. Advantageously, the peptides according to the invention have:

a mean hydrophobicity per residue <H> between 0.15 and 0.7;

a helical hydrophobic moment <μH>α greater than 0.15;

a beta hydrophobic moment <μH>β greater than 0.

The research work conducted for the invention related to three parameters used to orient the creation of primary sequences from reference sequences (Eisenberg et al., 1982; The helical hydrophobic moment: a measure of the amphilicity of helix. Nature 299, 371-374). The following three parameters were selected:

the mean hydrophobicity per residue,

the helical hydrophobic moment,

the beta hydrophobic moment.

The mean hydrophobicity per residue is a measure of the non-polar nature of a peptide. The hydrophobicity of a peptide is quantified by calculating the sum of the contribution of each residue to said hydrophobicity according to the formula:

$$⟨ H ⟩ = \frac{1}{N} {\sum }_{n = 1}^{N} {H}_{n}$$

wherein, H corresponds to the mean hydrophobicity per residue, N represents the number of residues, Hn represents the hydrophobic index of the residue n. The hydrophobic scale used is that of Fauchète and Pliska (1983, Eur. J. Med. Chem 18.369-375). The sum is calculated on all the residues of the helix (from n=1 to N).

The helical hydrophobic moment <μH>α is used to quantify the more or less amphiphilic nature of a helix a according to the formula:

$$⟨ μ H ⟩ = \frac{1}{N} {⟨ {[ {\sum }_{n = 1}^{N} {H}_{n} sin ( δn ) ]}^{2} + {[ {\sum }_{n = 1}^{N} {H}_{n} cos ( δn ) ]}^{2} ⟩}^{1 / 2}$$

wherein H corresponds to the hydrophobic index of the residue n and δ corresponds to the angle between 2 successive residues (100° for a helix α).

Each amino acid is assigned a carrier, the direction of which corresponds to the line connecting the centre of the wheel to its position on said wheel. The value of said carrier is equal to the hydrophobic index of the amino acid. The direction of the carrier goes from the centre to the amino acid if the amino acid is hydrophobic (positive value in hydrophobic scale) and from the amino acid to the centre if the amino acid is polar (negative value in hydrophobic scale).

The hydrophobic moment per amino acid of the amphiphilic helix is equal to the value of the carrier resulting from the sum of all said carriers, divided by the number of amino acids of the helix (from n=1 to N). The resulting carrier points in the direction of the most hydrophobic area of the wheel.

The beta hydrophobic moment <μH>β corresponds to the hydrophobic moment of a peptide which adopts a β leafed structure. The beta hydrophobic moment is calculated as for the <μH>α parameter but in this case, δ which corresponds to the angle between 2 successive residues, is equal to 180°.

The study of the three above-mentioned parameters was applied to three reference peptides:

two peptides derived from protegrin referred to as SynB1 and SynB3,

one peptide derived from tachyplesin referred to as SynB4.

The amino acid substitutions performed on the peptides SynB1, SynB3 and SynB4 reported below made it possible to identify sequence modifications resulting in highly amphipathic peptides.

The invention particularly relates to a peptide comprising or composed of a derivative of a peptide with the following amino acid sequence:

1 5 10 15 18
-R G G R L S Y S R R R F S T S T G R
1 5 10
-R R L S Y S R R R F
1 5 10 15 17
-A W S F R V S Y R G I S Y R R S R

or a fragment of said sequences composed of at least five and, preferentially, at least seven successive amino acids,

by substituting 1 to 18 and preferentially 1 to 6 amino acids and/or permuting at least one amino acid pair, said substitutions and/or permutation being such that said peptide is amphipathic in nature.

In the peptide sequences reported below, the amino acids are represented by their one-letter code, but they may also be represented by their three-letter code according to the list below.

| | | |
|---|---|---|
| A | Ala | alanine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| V | Val | valine |
| W | Trp | tryptophan |
| Y | Tyr | tyrosine |

1) Mutation of peptide SynB1.

1.1) Increase in hydrophobicity <H> of SynB1.

The following mutations increase the parameter <H>, i.e., the mean hydrophobicity per residue:

the mutation of at least one of the residues in position 6, 8, 13, 14, 15, 16 by a hydrophobic amino acid, preferentially selected from the group comprising: Ala, Ile, Leu, Val, Phe, Trp.

possibly the mutation of at least one other residue, preferentially the residue Phe in position 12, by a Tyr or Trp residue giving a spectroscopic signal and enabling the assay of the peptide.

Table 1 below gives the mutations enabling an increase in the hydrophobicity of SynB1 and gives examples of peptides according to the invention derived from SynB1.

TABLE 1

| | |
|---|---|
| <H> | <μH>α |

| | | | |
|---|---|---|---|
| SynB1 | R G G R L S Y S R R R F S T S T G R | −0.069 | 0.18 |
| | A A W A A A A | | |
| | I I I I I I | | |
| | L L L L L L | | |
| | V V V V V V | | |
| | F F F F F F | | |
| | W W W W W W | | |
| SM3979 | R G G R L A Y L R R R W A V L V G R | 0.295 | 0.27 |
| SM3980 | R G G R L V Y V R R R W V V V V G R | 0.343 | 0.25 |
| PG-4L | R G G R L L Y L R R R W L V L V G R | 0.45 | 0.27 |
| PG-4A | R G G R L A Y A R R R F A V A V G R | 0.115 | 0.2 |
| PG-AFL1 | R G G R L V Y L R R R F A F L I G R | 0.384 | 0.23 |

1.2) Increase in amphipathicity of SynB1.

The mutations below increase the parameters <H> and <μH>α, i.e., the mean hydrophobicity per residue and the mean helical amphipathicity per residue:

the permutation of at least one pair of residues in position 3 and 5, in position 10 and 12, and in position 16 and 17,

the mutation of at least one of the residues in position 6, 13, 14 and 16 with a hydrophobic amino acid, preferentially selected from the group comprising: Ala, Ile, Leu, Val, Phe, Trp,

possibly the mutation of at least one other residue, preferentially the residue Phe in position 12, by a Tyr or Trp residue giving a spectroscopic signal and enabling the assay of the peptide,

possibly the mutation of the residue in position 3 by a hydrophobic amino acid, preferentially selected from the group comprising: Ile, Leu, Val, Phe, Trp.

Table 2 below gives the mutations enabling an increase in the amphipathicity of SynB1 and gives examples of peptides according to the invention derived from SynB1.

TABLE 2

| | |
|---|---|
| <H> | <μH>α |

| | | | |
|---|---|---|---|
| SynB1 | R G G R L S Y S R R R F S T S T G R | −0.069 | 0.18 |
| A-SynB1 | R G L R G S Y S R F R R S T S T G R | −0.069 | 0.37 |
| | V A W A A A | | |
| | I V V V V | | |
| | F I I I I | | |
| | W L L L L | | |
| | F F F F | | |
| | W W W W | | |
| A-PG-3L | R G L R G L Y L R F R R L V S V G R | 0.327 | 0.43 |
| A-PG-IL | R G L R G L Y F R F R R I L S V G R | 0.364 | 0.45 |
| A-PG-4LF | R G I R G L Y L R W R R L L S F G R | 0.417 | 0.47 |
| A-PG-3-LF | R G F R G L Y L R W R R L V S V G R | 0.359 | 0.46 |

In Table 2 above, the peptide A-Synb1 is a derivative of SynB1 wherein the amino acid pairs in position 3 and 5, in position 10 and 12 and in position 16 and 17 have been permuted.

2) Mutation of peptide SynB3.

2.1) Increase in hydrophobicity <H> of SynB3.

The following mutations increase the parameter <H>, i.e. the mean hydrophobicity per residue:

the simultaneous mutation of the residues in position 4 and 6 or the simultaneous mutations of the residues in position 4, 5 and 6 by hydrophobic amino acids, preferentially selected from the group comprising: Ala, Ile, Leu, Val, Phe, Trp.

possibly the mutation of at least one other residue, preferentially the residue Phe in position 10, by a Tyr or Trp residue giving a spectroscopic signal and enabling the assay of the peptide.

Table 3 below gives the mutations enabling an increase in the hydrophobicity of SynB3 and gives examples of peptides according to the invention derived from SynB3.

TABLE 3

| | | |
|---|---|---|
| <H> | <μH>α | <μH>β |

| | | | | |
|---|---|---|---|---|
| SynB3 | R R L S Y S R R R F | −0.068 | 0.42 | 0.01 |
| | A A A | | | |
| | I I I | | | |
| | L L L | | | |
| | V V V | | | |
| | F F F | | | |
| | W W W | | | |
| SM4287 | R R L W Y L R R R F | 0.335 | 0.46 | 0.40 |
| SM4288 | R R L W L L R R R F | 0.409 | 0.39 | 0.34 |

2.2) Increase in helical amphipathicity per residue <PH>α of SynB3.

The following mutations increase the <H> and <μH>α parameters of the SynB3 peptide, i.e. the mean hydrophobicity per residue and the mean helical amphipathicity per residue of this peptide:

the permutation of the residues in position 5 and 7 and the simultaneous mutation, on the sequence resulting from the permutation, of the residues in position 4 and 6 or the residues in position 4, 6 and 7 with hydrophobic amino acids, preferentially selected from the group comprising: Ala, Ile, Leu, Val, Phe and Trp,

possibly the mutation of at least one other residue, preferentially the residue Phe in position 10, by a Tyr or Trp residue giving a spectroscopic signal and enabling the assay of the peptide.

Table 4 below gives the mutations enabling an increase in the mean hydrophobicity per residue and mean helical amphipathicity per residue of SynB3 and gives examples of peptides according to the invention derived from SynB3.

TABLE 4

| | | |
|---|---|---|
| <h> | <μH>α | <μH>β |

| | | | | |
|---|---|---|---|---|
| SynB3 | R R L S Y S R R R F | −0.068 | 0.42 | 0.01 |
| | A A A | | | |
| | I I I | | | |
| | L L L | | | |
| | V V V | | | |
| | F F F | | | |
| | W W W | | | |
| SM4289 | R R L W R L Y R R F | 0.335 | 0.82 | 0.4 |
| SM4290 | R R L W R L L R R F | 0.409 | 0.88 | 0.34 |

2.3) Increase in beta amphipathicity <μH>β of SynB3

The following mutations increase the <H> and <μH>β parameters of the SynB3 peptide, i.e. the mean hydrophobicity per residue and the mean beta amphipathicity per residue <μH>β of this peptide:

the permutation of the residues in position 2 and 3 and the permutation of the residues in position 5 and 8 and on the sequence resulting from the permutation, the simultaneous mutation of the residues in position 4 and 6 or the simultaneous mutation of the residues in position 4, 6 and 8 with hydrophobic amino acids, preferentially selected from the group comprising: Ala, Ile, Leu, Val, Phe and Trp,

possibly the mutation of at least one other residue, preferentially the residue Phe in position 10, by a Tyr or Trp residue giving a spectroscopic signal and enabling the assay of the peptide.

Table 5 below gives the mutations enabling an increase in the parameters <H> and <μH>β of the peptide SynB3 and gives examples of peptides according to the invention derived from SynB3.

TABLE 5

| | | |
|---|---|---|
| <H> | <μH>α | <μH>β |

| | | | | |
|---|---|---|---|---|
| SynB3 | R R L S Y S R R R F | −0.068 | 0.42 | 0.01 |
| | R L R S R S R Y R F | | | |
| | A A A | | | |
| | I I I | | | |
| | L L L | | | |
| | V V V | | | |
| | F F F | | | |
| | W W W | | | |
| SM4291 | R L R W R L R Y R F | 0.335 | 0.35 | 1.1 |
| SM4292 | R L R W R L R L R F | 0.409 | 0.36 | 1.1 |

3) Mutation of peptide SynB4.

3.1) Increase in hydrophobicity <H> of SynB3.

The following mutations increase the parameter <H>, i.e. the mean hydrophobicity per residue:

the simultaneous mutation of the residues in position 3, 7, 12, 16 by hydrophobic amino acids, preferentially selected from the group comprising: Ala, Ile, Leu, Val, Phe, Trp.

Table 6 below gives the mutations enabling an increase in the hydrophobicity <H> of SynB4 and gives examples of peptides according to the invention derived from SynB4.

TABLE 6

| | | <H> | <μH>α |
|---|---|---|---|
| SynB4 | A W S F R V S Y R G I S Y R R S R | 0.24 | 0.047 |
| | A A A A | | |
| | I I I I | | |
| | L L L L | | |
| | V V V V | | |
| | F F F F | | |
| | W W W W | | |
| SynB4-4A | A W A F R V A Y R G I A Y R R A R | 0.32 | 0.047 |
| SynB4-2AL | A W L F R V A Y R G I L Y R R A R | 0.49 | 0.047 |
| SynB4-FALF | A W F F R V A Y R G I L Y R R F R | 0.58 | 0.086 |

3.2) Increase in amphipathicity of SynB4.

The mutations below increase the parameters <H> and <μH>α, i.e., the mean hydrophobicity per residue and the mean helical amphipathicity per residue:

a permutation of the residues in position 12 and 14, the residues in position 16 and 17 and a simultaneous mutation, on the sequence resulting from the permutation, of the residues in position 3, 7, 14 and 17 with hydrophobic amino acids, preferentially selected from the group comprising: Ala, Ile, Leu, Val, Phe and Trp,

Table 7 below gives the mutations enabling an increase in the parameters <H> and <μH>α, i.e., the mean hydrophobicity per residue and the mean helical amphipathicity per residue, of SynB4 and gives examples of peptides according to the invention derived from SynB4.

TABLE 7

| | |
|---|---|
| <H> | <μH>α |

| | | | |
|---|---|---|---|
| SynB4 | A W S F R V S Y R G I S Y R R S R | 0.24 | 0.047 |
| aSynB4 | A W S F R V S Y R G I R Y S R R S | 0.24 | 0.18 |
| | A A A A | | |
| | V V V I | | |
| | I I I L | | |
| | L L L V | | |
| | F F F F | | |
| | W W W W | | |
| aSynB4-2AL | A W A F R V L Y R G I R Y L R R A | 0.49 | 0.41 |
| aSynB4-IVFA | A W I F R V V Y R G I R Y F R R A | 0.55 | 0.48 |
| aSynB4-FIIL | A W F F R V I Y R G I R Y I R R L | 0.67 | 0.55 |

In Table 7, aSynB4 is a peptide obtained with a permutation of the residues in position 12 and 14 and the residues in position 16 and 17.

The invention also relates to a linear peptide containing or composed of an antibiotic peptide derivative by :

modifying the cysteine residues such that said peptide is free of disulphide bridges,

substituting one or more arginines with citrulline or ornithine.

Indeed, substituting arginines with citrullines or ornithines makes it possible to reduce toxicity of the peptide resulting from arginine cationic nature. It is thus possible, to use a larger quantity of peptide carrier. However, advantageously, depending on the size and number of arginines in the original antibiotic peptide, the peptides according to the invention comprise at least another one, preferentially at least another two and particularly preferentially, at least another three arginines.

Examples of peptides according to the invention wherein one or more arginines are replaced by citrullines (Cit) include the SynB1, SynB3 and SynB4 derivatives in table 8 below.

TABLE 8

| | |
|---|---|
| SynB1 | R G G R L S Y S R R R F S T S T G R |
| SynB1/2Cit | R G G R L S Y S Cit Cit R F S T S T G R |
| SynB1/3Cit | R G G R L S Y S Cit Cit Cit F S T S T G R |
| SM3979 | R G G R L A Y L R R R W A V L V G R |
| SM3979/2Cit | R G G R L A Y L Cit Cit R W A V L V G R |
| SM3979/3Cit | R G G R L A Y L Cit Cit Cit W A V L V G R |
| SM3980 | R G G R L V Y V R R R W V V V V G R |
| SM3980/2Cit | R G G R L V Y V Cit Cit R W V V V V G R |
| SM3980/3Cit | R G G R L V Y V Cit Cit Cit W V V V V G R |
| SynB3 | R R L S Y S R R R F |
| SynB3/2Cit | R R L S Y S Cit Cit R F |
| SynB3/3CitA | R R L S Y S Cit Cit Cit F |
| SynB3/3CitB | R Cit L S Y S Cit Cit R F |
| SM4289 | R R L W R L Y R R F |
| SM4289/2Cit | R R L W R L Y Cit Cit F |
| SM4289/3Cit | R Cit L W R L Y Cit Cit F |
| SM4290 | R R L W R L L R R F |
| SM4290/2Cit | R R L W R L L Cit Cit F |
| SM4290/3Cit | R Cit L W R L L Cit Cit F |
| SynB4 | A W S F R V S Y R G I S Y R R S R |
| SynB4/2Cit | A W S F R V S Y R G I S Y Cit Cit S R |
| SynB4/3Cit | A W S F Cit V S Y R G I S Y Cit Cit S R |
| SynB4-FALF | A W F F R V A Y R G I L Y R R F R |
| SynB4-FALF/2Cit | A W F F R V A Y R G I L Y Cit Cit F R |
| SynB4-FALF/3Cit | A W F F Cit V A Y R G I L Y Cit Cit F R |

The peptides according to the invention may be prepared using a chemical or genetic engineering process, the retro form may be produced and they may comprise D form amino acids. The invention also relates to fragments of said peptides composed of at least five and preferentially at least seven successive amino acids of the above sequences.

The invention also relates to the use of said amphipathic linear peptides or analogues of said peptides to carry one or more active substances through the membrane of in vivo or in vitro cells for therapeutic or diagnostic applications. According to the invention, the term “carrying” refers to a process capable of passing through the cell membrane(s) and bringing said active substance to a target located in a cell compartment such as the cytoplasm or nucleus.

In this way, the invention relates to compounds formed from at least one amphipathic linear peptide described above bound directly or indirectly to at least one active substance. Said compounds may be represented by the following formula I:

A_____B (I)

wherein:

A represents an amphipathic peptide according to the invention,

B represents an active substance, and

the horizontal line represents the bond between the active substance and the amphipathic linear peptide.

The active substances of the invention particularly include proteins, such as polypeptides or peptides, antibodies or antibody components, nucleic acids and oligonucleotides or ribozymes or, of course, active chemical molecules to treat or prevent human or animal diseases, such as antitumoral, antiviral agents and the like. This list is merely exemplary; not exhaustive.

In the field of diagnostics, the active substance may be a radioactive marker, a colored marker or any other substance capable of detecting a metabolism or a disease.

The link between the carrier peptide and the active substance, represented by horizontal lines in formula I, may be produced using any acceptable bonding means given the chemical nature and dimensions in formula I compounds. The bonds may be covalent, hydrophobic or ionic, and may or may not be split in physiological media or inside cells. The bond may comprise one or more intermediate compounds (linker).

The link may be produced at any site of the peptide A, wherein functional groups such as —OH, —SH, —COOH, —NH2 are naturally present or have been introduced.

The linking positions for the active substances may be at the N-terminal or C-terminal ends or on the peptide's lateral chains. Similarly, the link may be produced at any site of the active substance B, wherein functional groups such as —OH, —SH, —COOH, —NH2 are naturally present or have been introduced.

Therefore, the invention also relates to pharmaceutical formulations comprising as an active agent an effective quantity of at least one formula I compound in an acceptable vehicle.

Other advantages and characteristics of the invention will be seen in the following examples relating to the preparation of amphipathic linear peptides, their link with an active substances and the penetration of this conjugate into cells.

1) Synthesis of peptides labelled with NBD fluorescent group.

The peptides were synthesised with the Fmoc-tBu strategy using an AMS 422 (ABIMED, Germany). The peptide sequences are given in Table I. The N-terminal was labelled by an NBD fluorescent group according to the procedure described by [Gazit, E. et al. (1995) Biochemistry 34, 11479-11488].

The peptide on resin is first treated with piperidine [20% (v/v) in DMF] to remove the Fmoc protective group on the N-terminal. NBD-C1 in dry DMF (5-fold excess) is then added in the presence of DIEA (2-fold excess) for 6 hours protected from light with stirring to selectively label the N-terminal group. The resin is then removed with DMF and treated with a deprotective mixture to detach the peptides from the resin and deprotect the lateral chains. The peptide was then purified by HPLC (reverse phase high performance liquid chromatography) (Water-prep LC 40, Water) with a 0.01% TFA/acetonitrile gradient. The purity of the peptides was measured with UV absorbance criteria at 220 nm and 460 nm and was 95%.

2) Preparation of peptides linked with doxorubicin.

The linking of doxorubicin on a peptide by means of a succinic linker was performed in 3 steps.

Succinic anhydride (1.1 eq, dissolved in DMF) was added to doxorubicin hydrochloride (1 eq), dissolved in dimethylformamide (DMF) in the presence of Diisopropylethylamine (DIEA, 2 eq). After an incubation period of 20 min at ambient temperature, the doxorubicin hemisuccinate formed was then activated by adding PyBOP (Benzotriazol-1-yl-oxopyrrolidinephosphonium Hexafluorophosphate 1.1 eq in DMF) and DIEA (2 eq). This second reaction mixture was incubated for 20 min. The peptide (1.2 eq in DMF) was then added to the reaction mixture and spontaneously bonded with the doxorubicin hemisuccinate activated during an additional incubation period of 20 min.

The linker was then purified on preparative HPLC (High Pressure Liquid Chromatography) and freeze-dried.

Each of the steps and the final product were tested using analytical HPLC and mass spectrometry.

The peptides tested are listed in Table 9 below.

TABLE 9

| | |
|---|---|
| Peptide | Sequence |

| | |
|---|---|
| SynB1 | RGGRLSYSRRRFSTSTGR |
| SM3979 | RGGRLAYLRRRWAVLVGR |
| SM3980 | RGGRLVYVRRRWVVVVGR |
| SM3505 (SynB3) | RRLSYSRRRF |
| SM4287 | RRLWYLRRRF |
| SM4288 | RRLWLLRRRF |
| SM4289 | RRLWRLYRRF |
| SM4290 | RRLWRLLRRF |
| SM4291 | RLRWRLRYRF |
| SM4292 | RLRWRLRLRF |
| SM4293 | RKLWYLRKRF |

3) Cell penetration.

Cell penetration of the peptides was studied by flow cytometry. The K562 cells were cultured in RPMI medium with 10% foetal calf serum. The cells were diluted to 0.3×10⁶ cells per ml 24 hours before the experiment. The cell penetration was measured by flow cytometry using a FACScan (Becton Dickinson, USA). The peptides labelled with NBD (final concentration 1 μM) were incubated with the K562 cells (5×10⁵ cells per ml) in Optimem medium at 37° C. for variable periods (the final volume was 0.5 ml). After incubation, the cells were washed twice and then resuspended in 0.5 ml of cold PBS. The penetration was then analysed by FACS. The fluorophors were excited at 488 nm and the fluorescence measured at 525 nm. A histogram of the fluorescent intensity (for 1×10⁴ cells) was obtained and the mean distribution considered to be representative of the quantity of peptide associated with the cells.

4) Results.

The internalisation results are listed in the tables below.

Table 10 shows the penetration of the peptide SM2363 with reference to that of two of its analogues which are more amphipathic.

TABLE 10

| Time (min) | SynB1 | SM3979 | SM3980 |
|---|---|---|---|
| 0 | 4 | 4 | 4 |
| 5 | 7 | 75 | 34 |
| 10 | 7 | 95 | 42 |
| 30 | 8 | 100 | 54 |
| 60 | 12 | 114 | 62 |

The results in Table 10 show that the amphipathic analogues (SM3979 and SM3980) penetrate much more effectively, irrespective of the time interval. In this way, at 60 min, the penetration of SM3979 and SM3980 is 9 and 5 times greater than that of SynB1, respectively.

The same design was carried out on shorter peptides. The peptide SynB3 was taken as the reference and its amphipathicity was increased. The comparison of the penetration of the peptide SynB3 with its analogues is represented in Tables 11 and 12 below.

TABLE 11

| Time (min) | SynB3 | SM4287 | SM4288 | SM4289 | SM4290 |
|---|---|---|---|---|---|
| 0 | 4 | 4 | 4 | 4 | 4 |
| 5 | 9.5 | 29 | 33 | 63 | 147 |
| 10 | 10 | 34 | 38 | 80 | 168 |
| 30 | 12 | 50 | 53 | 107 | 366 |
| 60 | 16 | 60 | 61 | 135 | 371 |

TABLE 12

| Time (min) | SynB3 | SM4291 | SM4292 | SM4293 |
|---|---|---|---|---|
| 0 | 4.5 | 4.5 | 4.5 | 4.5 |
| 5 | 12 | 91 | 140 | 20 |
| 10 | 13 | 99 | 140 | 23 |
| 30 | 15 | 107 | 141 | 29 |
| 60 | 23 | 118 | 146 | 31 |

The results in Tables 11 and 12 also demonstrate that the amphipathic analogues penetrate much more effectively, irrespective of the time interval.

5) Use of the peptides according to the invention as vectors for the transfer of an active molecule through the blood-brain barrier (BBB).

In numerous central nervous system diseases, the molecules administered do not pass through the blood-barrier and, therefore, are unable to reach their in the brain.

a) Experimental conditions

i) Preparation of SynB1/3Cit-Doxorubicin

The coupling of doxorubicin on the SynB1/3Cit peptide via the succinic link was performed in 3 steps:

Succinic anhydride (1.1 eq. dissolved in DMF) was added to doxorubicin hydrochloride (1 eq) and solubilised in dimethylformamide (DMF) in the presence of Disopropylethylamine (DIEA, 2 eq).

After incubation for 20 min at ambient temperature, the doxorubicin hemisuccinate formed was then activated by adding PyBOP (Benzotriazol-1-yl-oxopyrrolidinephosphonium Hexafluorophosphate, 1.1 eq in DMF) and DIEA (2 eq). This second reaction mixture was incubated for 20 min.

The SynB1/3Cit peptide (1.2 eq in DMF) was then added to the reaction mixture and coupled spontaneously with the activated doxorubicin hemisuccinate during an additional 20 min incubation period.

The coupling product was then purified in preparative HPLC (High Performance Liquid Chromatography), and freeze-dried.

Each of the steps, and the final product, were monitored by means of analytical HPLC and mass spectrometry.

ii) Products tested

| | |
|---|---|
| Doxo | Doxo |
| Doxo-SynB1/3Cit | Doxo-(RGGRLSYSCitCitCitFSTSTGR) |

iii) In situ brain infusion

This is a rapid and sensitive method to evaluate the penetration of various compounds in the central nervous system. Young mice (25-30 g, Iffa-Credo; l'Arbresle, France) were anaesthetised. After exposure of the common carotid, the right external carotid artery was tied at the bifurcation with the internal carotid and the common carotid was tied between the heart and the catheter implantation site (polyethylene catheter, ID: 0.76). The catheter previously filled with a heparin solution (100 units/ml) was inserted into the common carotid. The mice were infused with the infusion buffer (128 mM NaCl, 24 mM NaHCO₃, 4.2 mM KCl, 2.4 mM NaH₂PO₄, 1.5 mM CaCl₂, 0.9 mM MgSO₄ and 9 mM D-glucose). The buffer was filtered and then bubbled with a mixture containing 95% O₂/5% CO₂ to maintain the pH close to 7.4 and supply the brain with oxygen during the infusion.

The mice were infused with the buffer containing free doxorubicin or doxo-SynB1/3Cit. In each product, the doxorubicin was radiolabelled with carbon 14 (specific activity: 9.4 μCi/mg, Amersham, France) . The products were infused at a concentration of 0.33 μCi/ml or 0.035 mg/mouse.

Just before the start of infusion, the heart was stopped by ventricular section to prevent infusate reflux during infusion. The right hemisphere was then infused at a rate of 10 ml/min for 60 seconds, after which the mouse was decapitated.

b) In situ brain infusion results

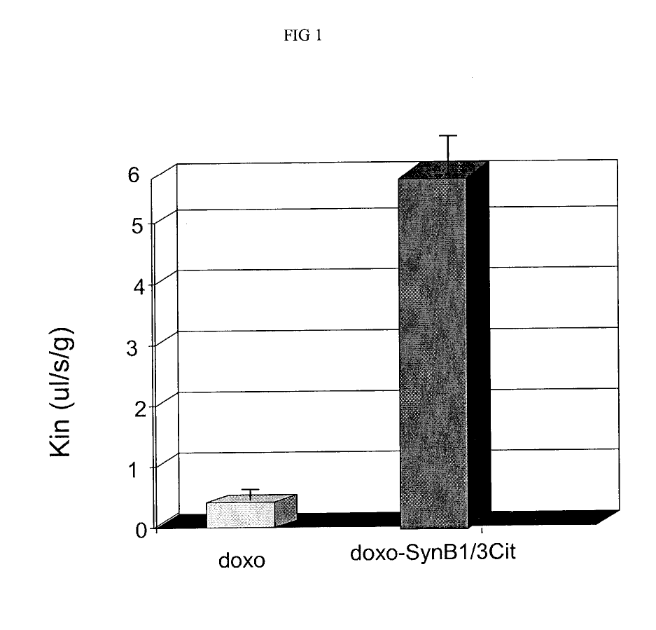

In this study, we compared the penetration in the BBB of doxorubicin alone with doxorubicin vectorised with SynB1/3Cit. After 60 seconds of infusion in the buffer, the penetration of the products was estimated by the influx constant or Kin in μg/sec/g. FIG. 1 shows that the vectorisation of doxorubicin with the vector SynB1/3Cit increases its passage into the brain 20 times after an infusion of 60 seconds in buffer.

1. A peptide comprising an antibiotic peptide derivative formed by:
modifying the cysteine residues such that the peptide is free of disulphide bridges, and
substituting 1 to 18 amino acids and/or permuting at least one amino acid pair, the substitutions and/or permutation being such that the peptide is amphipathic in nature, and has a mean hydrophobicity per residue <H> between 0.15 and 0.7.

2. The peptide according to claim 1, having a mean helical hydrophobic moment <μH>α greater than 0.15.

3. The peptide according to claim 1, having a beta hydrophobic moment <μH>β greater than 0.

4. The peptide according to claim 1, derived from protegrin or tachyplesin, from an analogue or a fragment thereof.

5. A peptide comprising a derivative of a peptide having an amino acid sequence is selected from the group consisting of:

| | |
|---|---|
| | 1 5 10 15 18 |
| SEQ ID NO: 1: | R G G R L S Y S R R R F S T S T G R |
| | 1 5 10 |
| SEQ ID NO: 2: | R R L S Y S R R R F |
| | 1 5 10 15 17 |
| SEQ ID NO: 3: | A W S F R V S Y R G I S Y R R S R |

or a fragment thereof composed of at least five and successive amino acids, by substituting 1 to 18 amino acids and/or permuting at least one amino acid pair, substitutions and/or permutation being such that peptide is amphipathic.

6. The peptide according to claim 5, derived from a peptide having an amino acid sequence SEQ ID NO:1 by mutation of at least one residue in positions 6, 8, 13, 14, 15, 16 with a hydrophobic amino acid, selected from the group consisting of: Ala, Ile, Leu, Val, Phe, Trp, and optionally mutation of at least one other residue selected from the group consisting of residue Phe in position 12, a Tyr residue and a Trp residue.

7. The peptide according to claim 6, further comprising at least one amino acid sequence selected from the group consisting of:
SEQ ID NO: 4:
R G G R L A Y L R R R W A V L V G R,
SEQ ID NO: 5:
R G G R L V Y V R R R W V V V V G R,
SEQ ID NO: 6:
R G G R L L Y L R R R W L V L V G R,
SEQ ID NO: 7:
R G G R L A Y A R R R F A V A V G R, and
SEQ ID NO: 8:
R G G R L V Y L R R R F A F L I G R.

8. The peptide according to claim 7, derived from a peptide wherein the amino acid sequence is SEQ ID NO:1, SEQ ID NO:4, SEQ ID NO:5, SEQ ID NO:6, SEQ ID NO:7 or SEQ ID NO:8 by permutation of at least one pair of residues in positions 3 and 5, positions 10 and 12 and positions 16 and 17.

9. The peptide according to claim 7, derived from a peptide wherein the amino acid sequence is SEQ ID NO:1, SEQ ID NO:4, SEQ ID NO:5, SEQ ID NO:6, SEQ ID NO:7 or SEQ ID NO:8 by:
permutation of at least one pair of residues in positions 3 and 5, positions 10 and 12, and positions 16 and 17,
mutation of at least one of residues in positions 6, 13, 14 and 16 with a hydrophobic amino acid, selected from the group consisting of: Ala, Ile, Leu, Val, Phe, Trp, and optionally mutation of at least one other residue selected from the group consisting of residue Phe in position 12, and a Tyr or Trp residue giving a spectroscopic signal and enabling an assay of the peptide, and
optionally the mutation of the residue in position 3 by a hydrophobic amino acid selected from the group comprising: Ile, Leu, Val, Phe, Trp.

10. The peptide according to claim 7, further comprising at least one amino acid sequence selected from the group consisting of:
SEQ ID NO: 9:
R G L R G S Y S R F R R S T S T G R,
SEQ ID NO: 10:
R G L R G L Y L R F R R L V S V G R,
SEQ ID NO: 11:
R G L R G L Y F R F R R I L S V G R,
SEQ ID NO: 12:
R G I R G L Y L R W R R L L S F G R, and
SEQ ID NO: 13:
R G F R G L Y L R W R R L V S V G R.

11. The peptide according to claim 5, derived from a peptide wherein the amino acid sequence is SEQ ID NO: 2 by simultaneous mutation of residues in positions 4 and 6 or simultaneous mutation of the residues in positions 4, 5 and 6 by hydrophobic amino acids selected from the group consisting of: Ala, Ile, Leu, Val, Phe, Trp, and optionally mutation of at least one other residue selected from the group consisting of residue Phe in position 10 and a Tyr or Trp residue giving a spectroscopic signal and enabling an assay of the peptide.

12. The peptide according to claim 11, further amino acid sequences:

| | |
|---|---|
| SEQ ID NO: 14: | R R L W Y L R R R F |
| SEQ ID NO: 15: | R R L W L L R R R F. |

13. The peptide according to claim 12, derived from a peptide wherein the amino acid sequence is SEQ ID NO: 2, SEQ ID NO: 14, SEQ ID NO: 15 by permutation of residues in positions 5 and 7 and simultaneous mutation, on a sequence resulting from the permutation, of residues in positions 4 and 6 or residues in positions 4, 6 and 7 with hydrophobic amino acids selected from the group consisting of: Ala, Ile, Leu, Val, Phe and Trp, and optionally mutation of at least one other residue selected from the group consisting of residue Phe in position 10, and a Tyr or Trp residue giving a spectroscopic signal and enabling an assay of the peptide.

14. The peptide according to claim 13, further comprising at least one amino acid sequence selected from the group consisting of:

| | |
|---|---|
| SEQ ID NO: 16: | R R L W R L Y R R F and |
| SEQ ID NO: 17: | R R L W R L L R R F. |

15. The peptide according to claim 14, derived from a peptide wherein the amino acid sequence is SEQ ID NO: 2, SEQ ID NO: 14, SEQ ID NO: 15, SEQ ID NO: 16 or SEQ ID NO: 17 by permutation of residues in positions 2 and 3 and permutation of the residues in positions 5 and 8 and on a sequence resulting from the permutation, the simultaneous mutation of residues in position 4 and 6 or simultaneous mutation of residues in position 4, 6 and 8 with hydrophobic amino acids selected from the group consisting of: Ala, Ile, Leu, Val, Phe and Trp, and optionally mutation of at least one other residue selected from the group consisting of residue Phe in position 10, and a Tyr or Trp residue giving a spectroscopic signal and enabling an assay of the peptide.

16. The peptide according to claim 15, further comprising at least one amino acid sequence selected from the group consisting of:

| | |
|---|---|
| SEQ ID NO: 18: | R L R W R L R Y R F |
| SEQ ID NO: 19: | R L R W R L R L R F |

17. The peptide according to claim 5, derived from a peptide wherein the amino acid sequence is SEQ ID NO: 3 by simultaneous mutation of at least one of residues in positions 3, 7, 12, 16 by hydrophobic amino acids selected from the group consisting of: Ala, Ile, Leu, Val, Phe and Trp.

18. The peptide according to claim 17, further comprising at least one amino acid sequence selected from the group consisting of:

| | |
|---|---|
| SEQ ID NO: 20: | A W A F R V A Y R G I A Y R R A R |
| SEQ ID NO: 21: | A W L F R V A Y R G I L Y R R A R |
| | and |
| SEQ ID NO: 22: | A W F F R V A Y R G I L Y R R F R. |

19. The peptide according to claim 18, derived from a peptide wherein the amino acid sequence in SEQ ID NO: 3, SEQ ID NO: 20, SEQ ID NO: 21 or SEQ ID NO: 22 by permutation of the residues in positions 12 and 14, residues in positions 16 and 17 and a simultaneous mutation, on a sequence resulting from the permutation, of the residues in positions 3, 7, 14 and 17 with hydrophobic amino acids selected from the group consisting of: Ala, Ile, Leu, Val, Phe and Trp.

20. The peptide according to claim 19, further comprising at least one amino acid sequence selected from the group consisting of:

| | |
|---|---|
| SEQ ID NO: 23: | A W A F R V L Y R G I R Y L R R A |
| SEQ ID NO: 24: | A W I F R V V Y R G I R Y F R R A, |
| | and |
| SEQ ID NO: 25: | A W F F R V I Y R G I R Y I R R L. |

21. The peptide according to claim 1, wherein one or more arginines are substituted by citrulline or ornithine.

22. The peptide according to claim 21, comprising at least one amino acid sequence slected from the group consisting of:
SEQ ID NO: 26:
R G G R L S Y S Cit Cit R F S T S T G R
SEQ ID NO: 27:
R G G R L S Y S Cit Cit Cit F S T S T G R
SEQ ID NO: 28:
R G G R L A Y L Cit Cit R W A V L V G R
SEQ ID NO: 29:
R G G R L A Y L Cit Cit Cit W A V L V G R
SEQ ID NO: 30:
R G G R L V Y V Cit Cit R W V V V V G R
SEQ ID NO: 31:
R G G R L V Y V Cit Cit Cit W V V V V G R
SEQ ID NO: 32:
R R L S Y S Cit Cit R F
SEQ ID NO: 33:
R R L S Y S Cit Cit Cit F
SEQ ID NO: 34:
R Cit L S Y S Cit Cit R F
SEQ ID NO: 35:
R R L W R L Y Cit Cit F
SEQ ID NO: 36:
R Cit L W R L Y Cit Cit F
SEQ ID NO: 37:
R R L W R L L Cit Cit F
SEQ ID NO: 38:
R Cit L W R L L Cit Cit F
SEQ ID NO: 39:
A W S F R V S Y R G I S Y Cit Cit S R
SEQ ID NO: 40:
A W S F Cit V S Y R G I S Y Cit Cit S R
SEQ ID NO: 41:
A W F F R V A Y R G I L Y Cit Cit F R, and
SEQ ID NO: 42:
A W F F Cit V A Y R G I L Y Cit Cit F R.

23. A compound formed from at least one peptide according to claim 1 bound directly or indirectly to at least one active substance.

24. A pharmaceutical formulation comprising as an active agent a therapeutically effective quantity of at least one compound according to claim 23 in an acceptable vehicle.


SEQUENCE LISTING (52 sequences)

SEQ ID NO: 1 - protein, 18 aa, Unknown Organism
  Description of Unknown Organism Peptide SynB1
RGGRLSYSRRRFSTSTGR

SEQ ID NO: 2 - protein, 10 aa, Unknown Organism
  Description of Unknown Organism Peptide SynB3
RRLSYSRRRF

SEQ ID NO: 3 - protein, 17 aa, Unknown Organism
  Description of Unknown Organism Peptide SynB4
AWSFRVSYRGISYRRSR

SEQ ID NO: 4 - protein, 18 aa, Artificial Sequence
  Description of Artificial Sequence Synthetic peptide SM3979
RGGRLAYLRRRWAVLVGR

SEQ ID NO: 5 - protein, 18 aa, Artificial Sequence
  Description of Artificial Sequence Synthetic peptide SM3980
RGGRLVYVRRRWVVVVGR

SEQ ID NO: 6 - protein, 18 aa, Artificial Sequence
  Description of Artificial Sequence Synthetic peptide PG-4L
RGGRLLYLRRRWLVLVGR

SEQ ID NO: 7 - protein, 18 aa, Artificial Sequence
  Description of Artificial Sequence Synthetic peptide PG-4A
RGGRLAYARRRFAVAVGR

SEQ ID NO: 8 - protein, 18 aa, Artificial Sequence
  Description of Artificial Sequence Synthetic peptide PG-AFL1
RGGRLVYLRRRFAFLIGR

SEQ ID NO: 9 - protein, 18 aa, Artificial Sequence
  Description of Artificial Sequence Synthetic peptide A-SynB1
RGLRGSYSRFRRSTSTGR

SEQ ID NO: 10 - protein, 18 aa, Artificial Sequence
  Description of Artificial Sequence Synthetic peptide A-PG-3L
RGLRGLYLRFRRLVSVGR

SEQ ID NO: 11 - protein, 18 aa, Artificial Sequence
  Description of Artificial Sequence Synthetic peptide A-PG-IL
RGLRGLYFRFRRILSVGR

SEQ ID NO: 12 - protein, 18 aa, Artificial Sequence
  Description of Artificial Sequence Synthetic peptide A-PG-4LF
RGIRGLYLRWRRLLSFGR

SEQ ID NO: 13 - protein, 18 aa, Artificial Sequence
  Description of Artificial Sequence Synthetic peptide A-PG-3LF
RGFRGLYLRWRRLVSVGR

SEQ ID NO: 14 - protein, 10 aa, Artificial Sequence
  Description of Artificial Sequence Synthetic peptide SM4287
RRLWYLRRRF

SEQ ID NO: 15 - protein, 10 aa, Artificial Sequence
  Description of Artificial Sequence Synthetic peptide SM4288
RRLWLLRRRF

SEQ ID NO: 16 - protein, 10 aa, Artificial Sequence
  Description of Artificial Sequence Synthetic peptide SM4289
RRLWRLYRRF

SEQ ID NO: 17 - protein, 10 aa, Artificial Sequence
  Description of Artificial Sequence Synthetic peptide SM4290
RRLWRLLRRF

SEQ ID NO: 18 - protein, 10 aa, Artificial Sequence
  Description of Artificial Sequence Synthetic peptide SM4291
RLRWRLRYRF

SEQ ID NO: 19 - protein, 10 aa, Artificial Sequence
  Description of Artificial Sequence Synthetic peptide SM4292
RLRWRLRLRF

SEQ ID NO: 20 - protein, 17 aa, Artificial Sequence
  Description of Artificial Sequence Synthetic peptide SynB4-4A
AWAFRVAYRGIAYRRAR

SEQ ID NO: 21 - protein, 17 aa, Artificial Sequence
  Description of Artificial Sequence Synthetic peptide SynB4-2AL
AWLFRVAYRGILYRRAR

SEQ ID NO: 22 - protein, 17 aa, Artificial Sequence
  Description of Artificial Sequence Synthetic peptide SynB4-FALF
AWFFRVAYRGILYRRFR

SEQ ID NO: 23 - protein, 17 aa, Artificial Sequence
  Description of Artificial Sequence Synthetic peptide aSynB4-2AL
AWAFRVLYRGIRYLRRA

SEQ ID NO: 24 - protein, 17 aa, Artificial Sequence
  Description of Artificial Sequence Synthetic peptide aSynB4-IVFA
AWIFRVVYRGIRYFRRA

SEQ ID NO: 25 - protein, 17 aa, Artificial Sequence
  Description of Artificial Sequence Synthetic peptide aSynB4-FIIL
AWFFRVIYRGIRYIRRL

SEQ ID NO: 26 - protein, 18 aa, Artificial Sequence
  Description of Artificial Sequence Synthetic peptide SynB/2cit
RGGRLSYSXXRFSTSTGR

SEQ ID NO: 27 - protein, 18 aa, Artificial Sequence
  Description of Artificial Sequence Synthetic peptide SynB1/3cit
RGGRLSYSXXXFSTSTGR

SEQ ID NO: 28 - protein, 18 aa, Artificial Sequence
  Description of Artificial Sequence Synthetic peptide SM3979/2cit
RGGRLAYLXXRWAVLVGR

SEQ ID NO: 29 - protein, 18 aa, Artificial Sequence
  Description of Artificial Sequence Synthetic peptide SM3979/3cit
RGGRLAYLXXXWAVLVGR

SEQ ID NO: 30 - protein, 18 aa, Artificial Sequence
  Description of Artificial Sequence Synthetic peptide SM3980/2cit
RGGRLVYVXXRWVVVVGR

SEQ ID NO: 31 - protein, 18 aa, Artificial Sequence
  Description of Artificial Sequence Synthetic peptide SM3980/3cit
RGGRLVYVXXXWVVVVGR

SEQ ID NO: 32 - protein, 10 aa, Artificial Sequence
  Description of Artificial Sequence Synthetic peptide SynB3/2cit
RRLSYSXXRF

SEQ ID NO: 33 - protein, 10 aa, Artificial Sequence
  Description of Artificial Sequence Synthetic peptide SynB3/3citA
RRLSYSXXXF

SEQ ID NO: 34 - protein, 10 aa, Artificial Sequence
  Description of Artificial Sequence Synthetic peptide SynB3/3citB
RXLSYSXXRF

SEQ ID NO: 35 - protein, 10 aa, Artificial Sequence
  Description of Artificial Sequence Synthetic peptide SM4289/2cit
RRLWRLYXXF

SEQ ID NO: 36 - protein, 10 aa, Artificial Sequence
  Description of Artificial Sequence Synthetic peptide SM4289/3cit
RXLWRLYXXF

SEQ ID NO: 37 - protein, 10 aa, Artificial Sequence
  Description of Artificial Sequence Synthetic peptide SM4290/2cit
RRLWRLLXXF

SEQ ID NO: 38 - protein, 10 aa, Artificial Sequence
  Description of Artificial Sequence Synthetic peptide SM4290/3cit
RXLWRLLXXF

SEQ ID NO: 39 - protein, 17 aa, Artificial Sequence
  Description of Artificial Sequence Synthetic peptide SynB4/2cit
AWSFRVSYRGISYXXSR

SEQ ID NO: 40 - protein, 17 aa, Artificial Sequence
  Description of Artificial Sequence Synthetic peptide SynB4/3cit
AWSFXVSYRGISYXXSR

SEQ ID NO: 41 - protein, 17 aa, Artificial Sequence
  Description of Artificial Sequence Synthetic peptide SynB4-FALF/2cit
AWFFRVAYRGILYXXFR

SEQ ID NO: 42 - protein, 17 aa, Artificial Sequence
  Description of Artificial Sequence Synthetic peptide SynB4-FALF/3cit
AWFFXVAYRGILYXXFR

SEQ ID NO: 43 - protein, 10 aa, Artificial Sequence
  Description of Artificial Sequence Synthetic peptide
RLRSRSRYRF

SEQ ID NO: 44 - protein, 17 aa, Artificial Sequence
  Description of Artificial Sequence Synthetic peptide aSynB4
AWSFRVSYRGIRYSRRS

SEQ ID NO: 45 - protein, 10 aa, Artificial Sequence
  Description of Artificial Sequence Synthetic peptide SM4293
RKLWYLRKRF

SEQ ID NO: 46 - protein, 18 aa, Artificial Sequence
  Description of Artificial Sequence Synthetic peptide
RGGRLXYXRRRWXXXXGR

SEQ ID NO: 47 - protein, 18 aa, Artificial Sequence
  Description of Artificial Sequence Synthetic peptide
RGXRGXYSRWRRXXSXGR

SEQ ID NO: 48 - protein, 10 aa, Artificial Sequence
  Description of Artificial Sequence Synthetic peptide
RRLXXXRRRF

SEQ ID NO: 49 - protein, 10 aa, Artificial Sequence
  Description of Artificial Sequence Synthetic peptide
RRLXYXXRRF

SEQ ID NO: 50 - protein, 10 aa, Artificial Sequence
  Description of Artificial Sequence Synthetic peptide
RLRXRXRXRF

SEQ ID NO: 51 - protein, 17 aa, Artificial Sequence
  Description of Artificial Sequence Synthetic peptide
AWXFRVXYRGIXYRRXR

SEQ ID NO: 52 - protein, 17 aa, Artificial Sequence
  Description of Artificial Sequence Synthetic peptide
AWXFRVXYRGIRYXRRX